(19) United States
(12) Patent Application Publication
(10) Pub. No.: US 2018/0353129 A1
(43) Pub. Date: Dec. 13, 2018

(54) Surgical Eye Mask With Oxygen Monitor

(71) Applicant: Blaine S. Cameron, Plantation, FL (US)

(72) Inventor: Blaine S. Cameron, Plantation, FL (US)

(21) Appl. No.: 15/620,824

(22) Filed: Jun. 13, 2017

(51) Int. Cl. A61B 5/00; A61B 5/1455; A41D 13/11; A61F 9/04

(52) U.S. Cl. CPC A61B 5/6803; A61B 5/14552; A41D 13/1184; A61F 9/04; A61B 5/14555; A61B 2505/05; A61B 2562/0238; A61B 2562/04; A61B 2562/222; A61B 2562/227; A41D 2500/52; A41D 13/1161

(57) ABSTRACT

A surgical eye mask device that comprises a frame assembly, the frame assembly includes a frame body, a left eye opening and a right eye opening, a left eye lens coupled to the left eye opening and a right eye opening coupled to the right eye opening, and an oximeter coupled with the left eye lens and the right eye lens for being capable of independently monitoring the oxygen saturation of a patient's left and right eyes intraoperatively.

### FIELD OF THE INVENTION

The present invention relates to a surgical eye mask, and more particularly to a surgical eye mask device with oxygen monitor for monitoring person's oxygen saturation (SO₂) and perfusion of the eye and optic nerve.

### BACKGROUND OF THE INVENTION

Postoperative visual loss (“POVL”) is a rare surgical complication particularly increased following spine, cardiac, cervical, head, and other orthopedic surgical procedures. The pathophysiology of POVL is not fully known but largely believed to be due to central retinal artery occlusion, ischemic optic neuropathy, and cerebral vision loss.

While POVL is rare, two potential causes include improper positioning of the patient and external compression of the eye. The eyes are believed to be particularly at risk during surgical procedures due to the prone position of the patient in the operating room necessary for lumbar and cervical operations.

One method of identifying blood oxygen saturation is described in U.S. Pat. No. 7,044,918 entitled Calculation of Physiological plethysmograph identification, Plethysmograph pulse recognition processor filed Oct. 27, 2004 in which a method for identifying pulses in a plethysmograph waveform is described.

### SUMMARY OF THE INVENTION

The present invention advantageously provides a surgical eye mask device for monitoring eyes intraoperatively to reduce and/or prevent postoperative visual loss associated with surgery, including spinal surgery, and further including posterior ischemic optic neuropathy, anterior ischemic optic neuropathy, central retinal artery occlusion post-operative blindness, and complications to the eyes due to the prone position during surgery.

According to an embodiment of the present invention, a principal object is to provide an eye mask capable of both protecting the eyes intraoperatively and also monitors the physiological eye and optic nerves during surgical procedures, thereby reducing incidence of post-operative blindness.

According to an embodiment of the present invention, another object is to provide an infra-red pulse oximetry on or about an eye shield for monitoring a patient's eyes during a surgical procedure. While the instant embodiment depicts the oximetry on the outside of the eye shield, it is within the spirit and scope of the instant invention to include an oximetry either integrated with the eye shield or inside of the eye shield.

According to an embodiment of the present invention, another object is to provide an infra-red pulse oximetery capable of noninvasively measuring hypovolemic conditions so as to titrate patient fluids.

Yet another object of the present invention is to provide an infra-red pulse oximeter capable of delivering and/or making PVI calucations utilizing a PI value defined by the formula PI=AC/DC. In an embodiment hereof, the PVI calculates the PI values resulting from acceptable plethysmograph pulses. For example, a red channel plethysmograph responsive to red wavelength LED is used to verify acceptable pulses in the IR channel for each of the patient's eyes intraoperatively. PVI values may be calculated from a sorted and trimmed buffer representing a sliding time window of PI values. The sort orders the PI values from the minimum PI at one end of a buffer to the maximum PI at the other end of the buffer to the maximum PI at the other end of the buffer. A predetermined number of both maximum and minimum PIs are deleted or otherwise ignored from each end of the buffer and the PVI may be calculated as: PVI=[(PIMAX−PIMIN)/PIMAX]×100. The PVI may be the PI variation expressed as a percentage of the difference between the maximum and minimum PIs remaining in the buffer. In an embodiment, a median PVI is calculated from PVIs stored in a second buffer. A PVI enabled physiological monitor advantageously provides a noninvasive numerical measure of hypovolemic conditions relating to the titrate patient fluids.

According to another embodiment, another object of the present invention is to provide a surgical eye mask device comprising a frame assembly that comprises a frame body, a left eye opening and a right eye opening, a left eye lens coupled to said left eye opening; a right eye opening coupled to said right eye opening, an oximeter coupled with said left eye lens and said right eye lens for being capable of independently monitoring left and right eyes of a patient intraoperatively. The oximeter includes a left infra-red light element and a right infra-red light element. The oximeter further includes a left infra-red detector sensor and a right infra-red detector sensor. The oximeter electrically is capable of coupling with a peripheral monitoring device via an intermediate cable.

Yet another object of the present invention is to provide a strap portion capable of securing said frame assembly against a face of a patient intraoperatively.

Yet another object of the present invention is to provide the frame assembly out of foam or any other materials capable of contacting a patient's face during an operation. The frame assembly is optionally formed of a flexible material. The frame assembly is optionally sized to cover the patient's eyes and/or cheek bones and/or eye sockets intraoperatively.

Yet another object of the present invention is to provide a fastening means for securement about said strap portion.

According to an object of the present embodiment, another object of the present invention is to provide an oximeter that is capable of independently detecting light reflected from a patient's right and left eyes. The oximeter optionally identifies plethysmograph waveforms for identifying data corresponding to physiological acceptable pulses.

According to an object of the present embodiment, another object of the present invention is to provide A surgical eye mask device comprises, a frame assembly that comprises a frame body, a left eye opening and a right eye opening, a left eye lens coupled to said left eye opening, a right eye opening coupled to said right eye opening, an oximeter coupled with said left eye lens and said right eye lens for being capable of independently monitoring left and right eyes of a patient intraoperatively, said oximeter capable of identifying pulses in a plethysmograph waveform for acquiring plethysmograph waveform data related to one or more signals received from a light-sensitive detector that detects light having a plurality of wavelengths transmitted through the left and right eyes of a patient carrying pulsing blood.

Other objects will become evident as the present invention is described in detail below.

### BRIEF DESCRIPTION OF THE DRAWINGS

Embodiments of the present application are described herein in which similar elements are given similar reference characters, and a more complete understanding of the present invention, and the attendant advantages and features thereof, will be more readily understood by reference to the following detailed description when considered in conjunction with the accompanying drawings wherein:

FIG. 1 is a front perspective view a surgical eye mask device with an associated monitor in accordance with the principles of one embodiment of the present embodiment;

FIG. 2 is a rear perspective view of the surgical eye mask device shown in FIG. 1; and

FIG. 3 is a cross-sectional view of the surgical eye mask taken along line A-A of FIG. 1.

### DETAILED DESCRIPTION OF THE INVENTION

The present invention advantageously provides a surgical eye mask device with oxygen monitor. The present invention contemplates various types of surgical eye mask device for measuring blood oxygen saturation.

Accordingly, the device and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

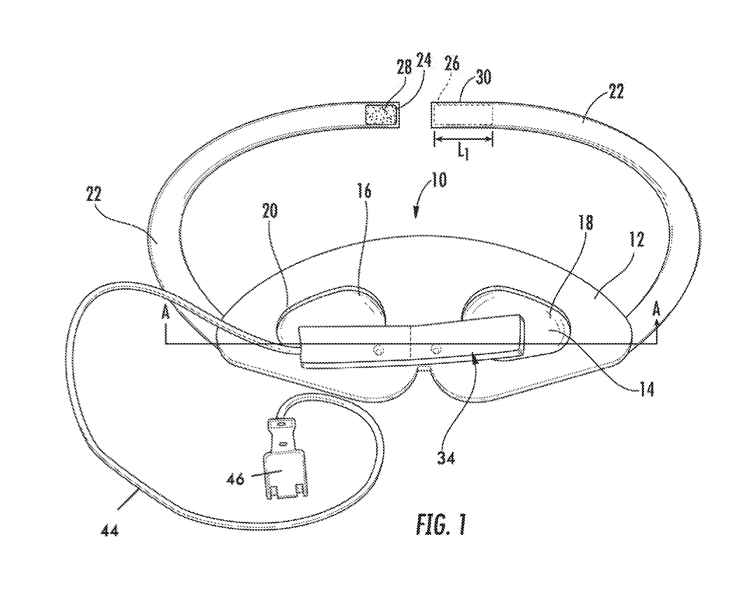

Referring now to the drawings figures in which like reference designators refer to like elements. FIG. 1 shows an exemplary surgical eye mask device constructed in accordance with the principles of the present invention and designated generally as surgical eye mask device 10. In particular, surgical eye mask device 10 comprises an eye frame assemblage 12. The frame assemblage comprises one or more eye openings, including a left eye opening 14 and a right eye opening 16. Corresponding to the left eye opening 14 is a left lens 18 secured about the frame assemblage 12. Corresponding to the right eye opening 16 is a right lens 20 secured about the frame assemblage 12. The left and rights lenses 18, 20 may be secured about the frame assemblage 12 in any for coupling the left and right lenses 18, 20 to the frame assemblage 12. Secured about the left and rights lenses 18, 20 is an oximeter probe assembly 34.

The frame assemblage 12 is optionally a soft and flexible material for preventing putting preserve or stress on a patient's face that could lead to postoperative visual loss. In one non-limiting embodiment, the frame assemblage 12 can optionally be constructed from foam.

The frame assemblage includes a strap portion 22 are combined with the frame assemblage 12. The strap portion 22 may be formed of any materials and may be attached to the frame assemblage 12. In one embodiment, the strap portion 22 may combine with the frame assemblage 12 in a one-piece formation. The strap portion 22 has two distal ends 24 and 26 that includes a fastening means 28 and 30. In one non-limiting embodiment, the fastening means 28, 30 is reciprocating hook and loop material. The fastening means 28, 30 can alternatively be any type of fastening means, including without limitation snaps, hooks, zippers, magnetic fastening means, or other like fastening means. The fastening means 30 may have a length L1 for allowing user adjustability of the mask for varying the tension of the surgical eye mask device about the face of a patient due to the varying face and head sizes of different patients.

In one non-limiting alternative embodiment, the frame assemblage 12 includes at least one opening for at least one single transparent lens.

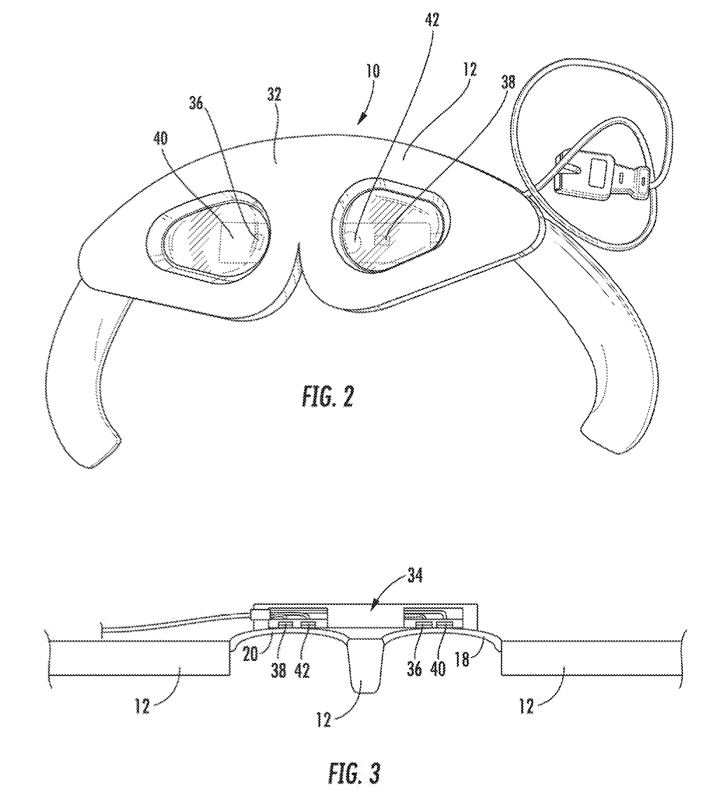

Referring now to FIG. 2 showing the surgical eye mask device 10, the frame assemblage 12 includes a foam inner surface capable of contacting a patient's face during surgical operations. The oximeter probe assembly 34 includes a left light emitting element 36 and a right light emitting element 38. The left light emitting element 36 directs light into the left eye opening 18 towards a patient's left eye. The right light emitting element 38 directs light into the right eye opening 16 towards a patient's right eye. The oximeter probe assembly 34 further includes a left light detecting sensor 40 and a right light detecting sensor 42, the light detecting sensors 40, 42 detect infra-red light emitted from the left and right light emitting elements 36, 38 reflected back from a patient's left and right eyes, respectively. The oximeter probe assembly 34 detects independently the blood oxygen saturation for each of the left and right eyes of a patient wearing the surgical eye mask device 10 during surgery.

Referring now to FIG. 3, a cross-sectional view taken along line A-A of FIG. 1 is shown of portions of the frame assemblage 12 coupled with the left and rights lens 18, 20 and also coupled with the oximeter probe assembly 34, depicting the left and right light emitting elements 36, 38, and the left and right light detecting sensors 40, 42.

Referring now to FIGS. 1 and 3, wiring 44 extends to a proximal plug 46. The proximal plug 46 is capable of connecting with a peripheral monitor device for interpreting and monitoring the oxygen saturation of a patient at or near each of the patient's left and right eye during a surgical procedure and/or operation. The proximal plug 46 is further capable of providing power for powering the left and right light emitting elements 36 and 38.

A plethysmograph pulse recognition processor may be utilized according to U.S. Pat. No. 7,044,918, entitled Plethysmograph Pulse Recognition Processor, which is hereby incorporated by reference in its entirety. PVI is described in U.S. Pat. No. 8,414,499 entitled “Plethysmograph Variability Index,” which is hereby incorporated by reference in its entirety.

As used herein, when the term “and/or” is used, it shall include all combinations of one or more of the associated described items.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are contemplated in light of the above teachings without departing from the scope and spirit of the invention. It will be readily apparent that various changes may be made thereto without departing from the spirit and scope of the disclosure or sacrificing all of its material advantages. The examples and embodiments described herein are merely exemplary of the instant disclosure.

What is claimed is:

1. A surgical eye mask device comprising:
a frame assembly that comprises a frame body, a left eye opening and a right eye opening;
a left eye lens coupled to said left eye opening;
a right eye opening coupled to said right eye opening; and
an oximeter coupled with said left eye lens and said right eye lens for being capable of independently monitoring left and right eyes of a patient intraoperatively.

2. The surgical eye mask device of claim 1 wherein said oximeter includes a left infra-red light element and a right infra-red light element.

3. The surgical eye mask device of claim 2 wherein said oximeter further includes a left infra-red detector sensor and a right infra-red detector sensor.

4. The surgical eye mask device of claim 3 wherein said oximeter electrically capable of coupling with a peripheral monitoring device via an intermediate cable.

5. The surgical eye mask device of claim 3 wherein said frame assembly includes a strap portion capable of securing said frame assembly against a face of a patient intraoperatively.

6. The surgical eye mask device of claim 5 wherein said frame assembly is formed of foam.

7. The surgical eye mask device of claim 5 wherein said frame assembly is formed of a flexible material.

8. The surgical eye mask device of claim 5 wherein said frame assembly is sized to cover the patient's eyes intraoperatively.

9. The surgical eye mask device of claim 5 further comprising a fastening means secured about said strap portion.

10. The surgical eye mask device of claim 3 wherein said oximeter is capable of independently detecting light reflected from a patient's right and left eyes.

11. The surgical eye mask device of claim 1 wherein said oximeter identifies plethysmograph waveforms for identifying data corresponding to physiological acceptable pulses.

12. A surgical eye mask device comprising:
a frame assembly that comprises a frame body, a left eye opening and a right eye opening;
a left eye lens coupled to said left eye opening;
a right eye opening coupled to said right eye opening; and
an oximeter coupled with said left eye lens and said right eye lens for being capable of independently monitoring left and right eyes of a patient intraoperatively, said oximeter capable of identifying pulses in a plethysmograph waveform for acquiring plethysmograph waveform data related to one or more signals received from a light-sensitive detector that detects light having a plurality of wavelengths transmitted through the left and right eyes of a patient carrying pulsing blood.
